(19) United States
(12) Patent Application Publication
(10) Pub. No.: US 2003/0050676 A1
(43) Pub. Date: Mar. 13, 2003

(54) Suspension of implantable device

(76) Inventors: Mark Hubelbank, Sudbury, MA (US); Enrique Malaret, Plymouth, MN (US); Bruce D. Gunderson, Plymouth, MN (US); Walter H. Olson, North Oaks, MN (US); James H. Ericksen, Roseville, MN (US); Kevin T. Ousdigian, St. Paul, MN (US); Bradley A. Stone, Centerville, OH (US); Vincent W. Kapral, Campbell, CA (US)

Correspondence Address: MEDTRONIC, INC., 710 MEDTRONIC PARKWAY NE, MS-LC340, MINNEAPOLIS, MN, 55432-5604, US

(21) Appl. No.: 09/952,886

(22) Filed: Sep. 11, 2001

(51) Int. Cl. A61N 1/08

(52) U.S. Cl. 607/60

(57) ABSTRACT

In general, the invention is directed to techniques for using a magnet to suspend a function of an implanted device, and to verify that the function has been suspended. A suspension device emits a magnetic field to suspend a function of a device implanted in a body, receives a signal that the function has been suspended, and outputs an indicator that the function has been suspended. In this manner, an operator can proceed having confidence that the suspension of the function was completed, and did not fail due to inappropriate placement of the magnet, slippage, or any other of a number of reasons.

### FIELD

The invention relates to medical devices, and in particular to devices that communicate with implanted medical devices.

### BACKGROUND

In ordinary use, an implantable cardioverter/defibrillator (ICD) continually receives electrical signals from a patient's heart and processes the signals to monitor the patient's cardiac rhythms. The ICD typically includes leads that extend into the patient's heart, and support one or more defibrillation electrodes. When the ICD detects an abnormal cardiac rhythm, the ICD may deliver a high voltage shock to the heart via a defibrillation electrode to restore normal cardiac rhythm.

In some circumstances, an ICD may be at risk of providing inappropriate shocks. The ICD may provide inappropriate shocks when the ICD falsely detects an abnormal cardiac rhythm. This may occur, for example, when the patient undergoes surgery and may be subjected to electrocautery, diathermy or other procedure that employs the application of electromagnetic energy. Such procedures may generate electric signals that interfere with the ICD's detection and analysis of cardiac signals.

Interference resulting from such a procedure may have many adverse effects. The interference may cause the ICD to prematurely begin a blanking interval, for example, and as a result, the ICD may ignore legitimate cardiac signals. The interference may cause the ICD to detect an arrhythmia that is not in fact present, and, in response to the falsely detected arrhythmia, the ICD may deliver an undesired defibrillation pulse. Furthermore, not only may the undesired defibrillation pulse provide inappropriate therapy for the patient, the pulse may subject medical providers to an electric shock.

Accordingly, it is advantageous to disable defibrillation functions in surgical procedures that employ the application of electromagnetic energy. One way to disable defibrillation functions is to temporarily deactivate the ICD's arrhythmia detection function. Therefore, conventional ICDs often incorporate a magnetically operable switch that controls one or more activities of the implanted devices. When an operator places a magnet in proximity to the device, the magnet opens or closes the magnetically operable switch, which affects a function of the device. An ICD, for example, may include a magnetically operable switch that enables and disables signal detection or other functions.

In particular, some ICDs include a reed switch, Hall switch or other magnetically operable switch coupled to the circuitry that controls detection function. Placing a magnet in proximity to the ICD activates the switch and temporarily deactivates the arrhythmia detection function of the ICD, and thereby prevents the application of defibrillation pulses. When the magnet is moved away from the ICD, the arrhythmia detection function is re-enabled.

The deactivation of functions with a magnet is generally a temporary deactivation, i.e., a suspension of the function. Consequently, a magnet is not ordinarily used to turn off functions such as an arrhythmia detection function for an extended period. To deactivate such a function for an extended period, a device called a “programmer” is usually employed to program the ICD to turn the function off. A programmer is typically operated by a specialist.

The use of a magnet to suspend a function of an implantable device is simpler than calling in a specialist to program the ICD to deactivate and activate the ICD's detection function. The use of a magnet, however, may have several disadvantages. Inappropriate placement of the magnet, or slippage of an appropriately placed magnet, may result in a failure to suspend the desired function. In addition, some ICDs may be programmed to ignore the magnet, but the operator may be unaware of this programming.

### SUMMARY

In general, the invention is directed to techniques for using a magnet to suspend a function of an implanted device, and to verify that the function has been suspended.

In one embodiment, the invention presents a method comprising emitting a magnetic field to suspend a function of an implanted device and receiving a signal that the function has been suspended. When used in conjunction with a typical ICD, for example, the received signal may be generated by a telemetry system of the ICD in response to the suspension of the function. The method also comprises outputting an indicator that the function has been suspended, such as by illuminating a light.

In another embodiment, the invention presents a device comprising a magnet that produces a magnetic field, a receiver that receives a signal when an implanted device suspends a function in response to the magnetic field, and an indicator controlled by the receiver according to the signal. The receiver may include signal processing elements, such as an amplifier, a filter, a signal detector and a signal validator.

The invention provides many advantages, including providing confirmation to an operator that a function of the implanted device has indeed been suspended. In this manner, the operator can proceed having confidence that the suspension of the function was completed, and did not fail due to inappropriate placement of the magnet, slippage, or any other of a number of reasons.

Another advantage of the invention is ease of use. It is not necessary to call in a specialist with a programmer to confirm that the function of the implanted device has indeed been suspended. The operator may place the magnet proximal to the implanted device and receive an easy-to-understand confirmation, such as the illumination of a light. Should the patient require immediate re-enablement of the function, the operator may re-enable the function right away simply by moving the magnet away from the implanted device.

A further advantage is that the function is suspended, not turned off. The function is automatically re-enabled when the operator moves the magnet away. The invention eliminates the risk of having a function turned off and inadvertently not turned on again.

The above summary of the invention is not intended to describe every embodiment of the invention. The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a diagram illustrating an device, including a defibrillator, implanted in a body of a patient.

FIG. 2 is a block diagram of art implantable ICD interacting with a suspension device.

FIG. 3 is a block diagram of a suspension device.

FIG. 4 is an exploded perspective view of a suspension device.

FIG. 5 is a flow diagram illustrating an embodiment of the invention.

FIG. 6 is a flow diagram illustrating another embodiment of the invention.

FIG. 7 is a flow diagram illustrating a further embodiment of the invention.

### DETAILED DESCRIPTION

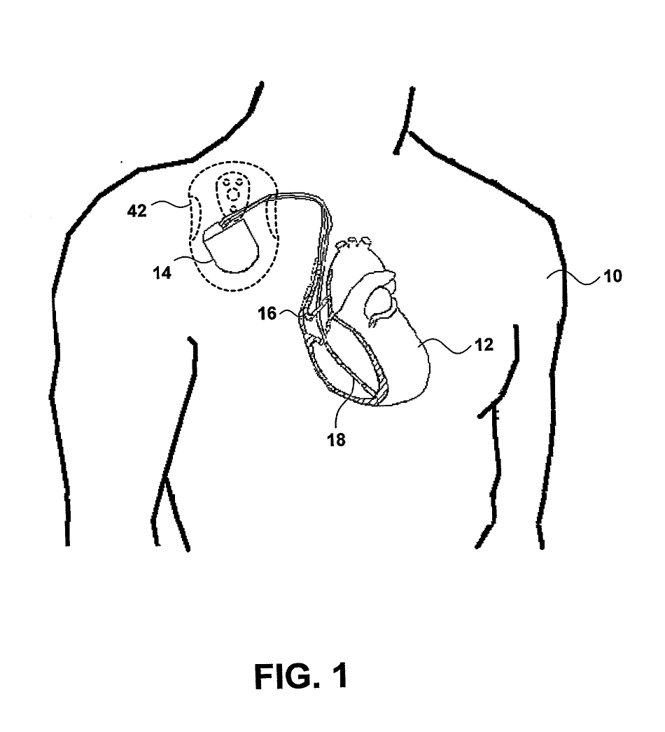

FIG. 1 shows a typical placement of an exemplary implanted medical device. In particular, FIG. 1 illustrates ICD 14 implanted within patient 10. For exemplary purposes, the invention is described with reference to ICD 14, but the invention is not limited to practice with cardioverter/defibrillators.

Leads 16 and 18 extend from ICD 14 into heart 12 of patient 10. Leads 16 and 18 may enter the vascular system at any of a number of entry sites, such as the cephalic vein. Lead 16 is disposed in the right atrium and lead 18 is disposed in the right ventricle. Lead 16 or lead 18 or both may be equipped with defibrillation electrodes, under the control of ICD 14.

Leads 16 and 18 may also include sensing electrodes, which sense the electrical activity of heart 12. A procedure that delivers electromagnetic energy to patient 10, such as electrocautery, may also be detected by leads 16 and/or 18.

ICD 14 is implanted near the right shoulder of patient 10. This implantation site is one of many implantation sites, and the invention is not limited to use at this site. Moreover, the implantation may be implanted below the skin, or below one or more muscles, such as the pectoral muscle. The invention may be practiced with implanted medical devices at a variety of depths.

FIG. 1 shows the placement of typical suspension device 42 near ICD 14. As described in detail below, ICD 14 suspends one or more functions in response to a magnetic field emitted by suspension device 42. Upon sensing the magnetic field and suspending a function, ICD generates a signal confirming the suspension of the function. For example, a telemetry system of the ICD may generate the signal. Upon receiving the signal, suspension device 42 outputs an indicator that the function has been suspended, such as by illuminating a light.

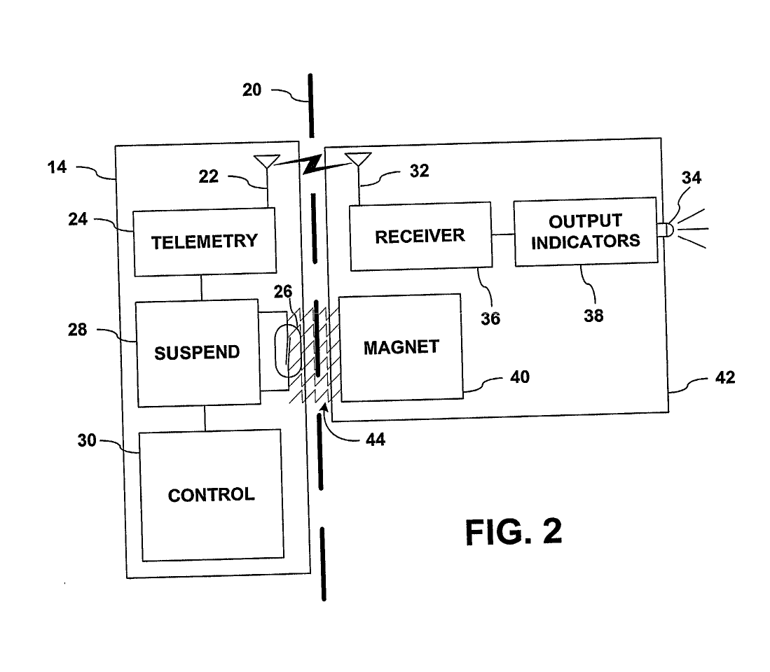

FIG. 2 is a block diagram illustrating an example system in which suspension device 42 interacts with ICD 14 to suspend a function, and provide confirmation of the suspension. In the illustrated embodiment, suspension device 42 includes magnet 40, which emits magnetic field 44 that passes through tissue barrier 20 and interacts with magnetically operable switch 26 in ICD 14. Magnetically operable switch 26, which may be a reed switch, a Hall effect switch or other suitable switch, opens or closes in response to a magnetic field emitted by magnet 40. Suspend circuitry 28 is coupled to magnetically operable switch 26, and in response to the change in state of magnetically operable switch 26, disables a function of ICD 14.

In a typical ICD, control circuitry 30 controls delivery of defibrillation pulses and other function. In addition, control circuitry 30 regulates the analysis of electric signals received via leads 16 and 18, and controls the timing of the delivery of defibrillation or cardioversion pulses. In a pacemaker/cardioverter/defibrillator, control circuitry 30 may also control basic time intervals associated with various modes of single and/or dual chamber pacing.

In response to a signal from suspend circuitry 28, control circuitry 30 suspends detection functions. Control circuitry 30 may, for example, temporarily disable the algorithms used to analyze electrical signals.

In response to a signal from suspend circuitry 28, telemetry element 24 generates a signal that is typically a coded signal indicating that the function has successfully been suspended. Antenna 22 transmits the coded signal, which transits tissue barrier 20.

Antenna 32 in suspension device 42 receives the transmitted coded signal from ICD 14. As will be described in more detail below, receiver 36 receives and validates the signal. Upon validating that the signal from ICD 14 confirms the suspension of the function, receiver 36 activates output indicators 34, generating an output indicating that the function is disabled. Receiver 36 may, for example, illuminate light 34, activate a tone generator (not shown in FIG. 2), or any other output suitable to provide an operator with confirmation of the suspension.

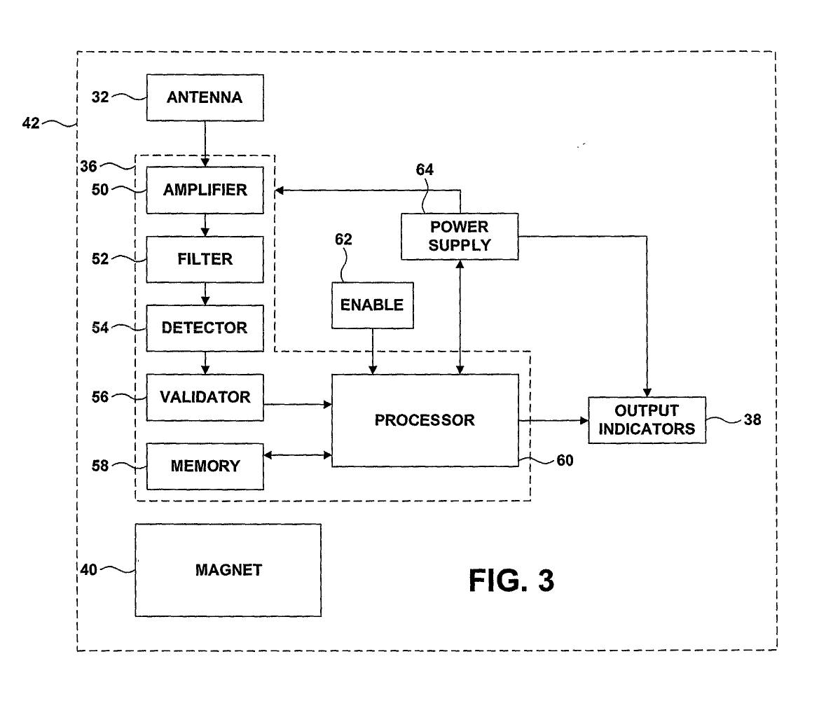

FIG. 3 is a block diagram showing an example implementation of suspension device 42. Receiver 36 receives a signal from antenna 32 and processes the signal to confirm that ICD 14 has indeed suspended the function. In particular, amplifier 50 and filter 52 amplify and filter the signal. Detector 54 senses whether a signal has been received, and validator 56 verifies and decodes the signal. When the decoded signal confirms that the function of ICD 14 has been suspended, validator 56 confirms to processor 60 that the function of ICD 14 has been suspended.

Upon receiving confirmation that the function of ICD 14 has been suspended, processor 60 may control output indicators 38 to generate an indication that the function of ICD 14 has been suspended. Similarly, when processor 60 fails to receive such confirmation, processor 60 may control output indicators 38 to generate an indication that the function of ICD 14 has not been suspended. In this way, a positive indication of a suspension of function will not be provided unless a signal from ICD 14 affirmatively acknowledges that the function has been suspended. Processor 60 typically operates in response to computer-readable instructions stored in memory 58. Memory 58 may comprise, for example, random access memory, read-only memory, or erasable programmable read-only memory.

Power supply 64 supplies power to receiver 36 and output indicators 38.

Magnet 40 is usually a permanent magnet and not dependent upon power supply 64. To conserve power, suspension device 42 may operate in a plurality of power modes. When off, suspension device 42 consumes no power and provides no indication of the state of the function of ICD 14. When activated by enabling mechanism 62 such as a push-button activation switch, suspension device 42 may operate in different power modes. For example, suspension device 42 may assume a high-power state when receiving signals from ICD 14. Alternatively, suspension device 42 may alternate rapidly between a high-power state, in which suspension device 42 listens for transmissions, and a low-power state, in which powered operations are powered down and listening is suspended. As another alternative, suspension device 42 may operate in a high-power mode while receiving signals from ICD 14, but when suspension device 42 fails to receive signals from ICD 14 for a period of time, suspension device 42 may assume a low-power standby mode. The present invention encompasses all of these alternatives.

In addition, power supply 64 communicates with processor 60 so that processor 60 may track the condition of power supply 64. If power supply 64 comprises batteries, for example, processor 60 may control output indicators 38 to generate an indication when battery power is low.

The elements shown in FIG. 3 are not the only embodiment of suspension device 42. The elements are depicted as logical entities and need not be built as separate elements. For example, amplifier 50 and filter 52 may be implemented in a single circuit, or the operations of detector 54 and validator 56 may be performed by processor 60.

In addition, suspension device 42 may include other elements not shown in FIG. 3. Power supply 64 may include a converter, for example, to provide a desired regulated voltage. Receiver 36 may include, for example, a peak detector, an analog-to-digital converter, or a cyclical redundancy checker. The signal processing elements of receiver 36 may depend upon the nature of the telemetry signal transmitted by ICD 14.

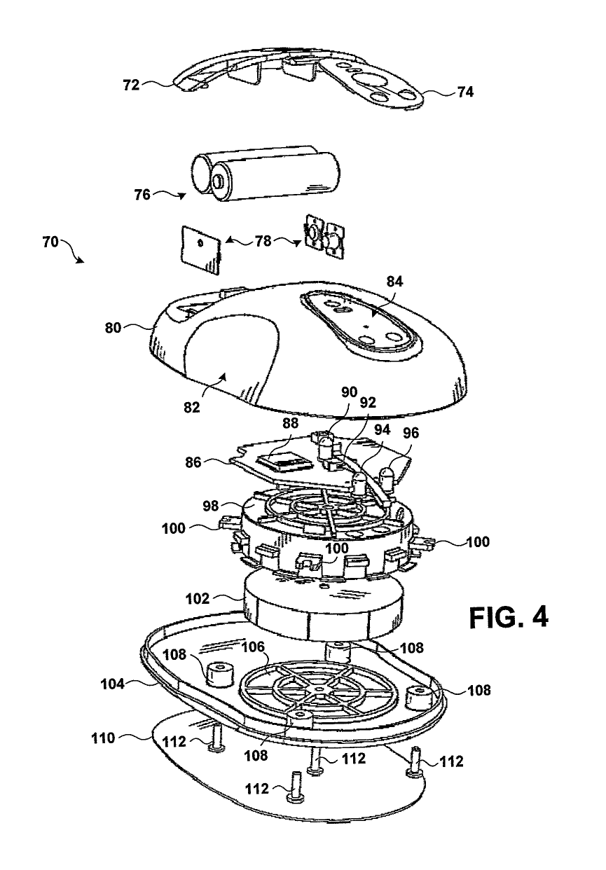

FIG. 4 is an exploded view of an implementation of the invention. Suspension device 70 includes a housing comprising base 104 and cover 80. Base 104 and cover 80 may be formed of molded plastic.

Permanent magnet 102 is disposed inside magnet housing 98, which fits over pedestal 106. Magnet housing 98 may also be formed of molded plastic, and may be held in place by anchor structures 100, which are secured by fasteners 112. Holding magnet 102 securely in place reduces the risk that the magnetic field will change position due to a shift of magnet 102 inside suspension device 70. Fasteners 112, which may be screws, feed through sockets 108 in base 104, engage anchor structures 100, and couple cover 80 to base 104 in the final assembly.

Base plate 110 may be secured to base 104 with adhesive. Base plate may be, for example, a softer plastic. Base plate 110 protects the patient from structures such as screw heads that may cause discomfort. Base plate 110 further may provide a non-slippery or tacky surface that contacts the patient's skin, making suspension device 70 less likely to slip when placed on the patient's skin. Once placed in an appropriate site on the patient, suspension device 70 may be secured in place with adhesive tape.

Magnet housing 98 supports circuit board 86. Circuit board 86 supports the electronic components described above in connection with FIG. 3, such as circuitry 88. Circuitry 88 may include components such receiver 42. Circuit board 86 may support electronic components in addition to those shown in FIG. 4.

Output indicators 90, 94 and 96, embodied as light-emitting diodes (LED's), are mounted to circuit board 86. Each of the three LED's may be assigned a dedicated function. For example, indicator 90 may be a red LED that illuminates when batteries 76 are low. Indicator 94 may be a green LED that illuminates when suspension device 70 receives confirmation that the function of ICD 14 has been suspended. Indicator 94 may be a yellow LED that illuminates when suspension device 70 is on and no confirmation has been received that the function of ICD 14 has been suspended.

Circuit board 86 supports switch 92, which may be spring-loaded. Switch 92, when depressed, turns on suspension device 70.

Cover 80 includes instrumentation outlet 84. Switch 92 may be accessed through instrumentation outlet 84, and output indicators 90, 94 and 96 may be seen. Switch 92 and output indicators 90, 94 and 96 may be protected by instrumentation cover 74.

Cover 80 may be include structures, such as indentation 82, that make it easier for an operator to handle suspension device 70. Cover 80 also includes a battery compartment (not shown in FIG. 4) that holds batteries 76. Conducting terminals 78 may be mounted in the battery compartment to hold batteries 76 in series. Battery compartment cover 72 snaps in place to cover the battery compartment.

Suspension device 70 is designed to be small enough to handle, yet large enough to be seen easily. Being large enough to be seen offers at least two advantages. First, output indicators 90, 94 and 96 are easily seen and distinguished. Second, suspension device 70 is not likely to be placed in proximity to the patient's ICD and inadvertently forgotten.

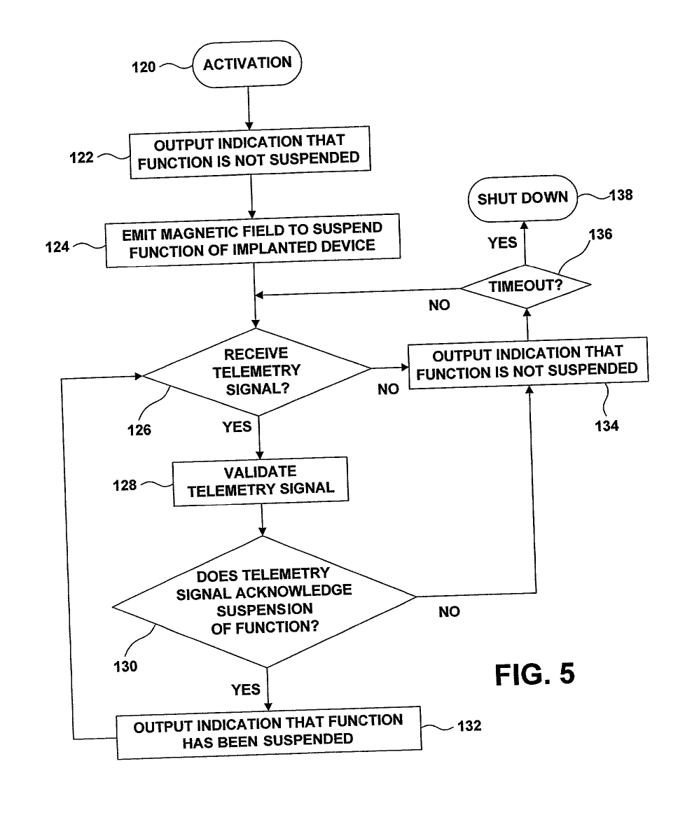

FIG. 5 is a flow diagram illustrating an example mode of operation of a suspension device according to the principles of the invention. In a typical application, when suspension device is, activated (120), the suspension device indicates that there has been no confirmation that the function of the implanted device has been suspended (122). In suspension device 70 shown in FIG. 4, suspension device 70 would indicate a lack of confirmation by illuminating yellow LED 96.

In the case of a suspension device having a permanent magnet, the device continually emits a magnetic field. Once moved into position relative to an implanted device, the suspension device emits a magnetic field to suspend a function of the implanted device (124). Exemplary techniques for suspending functions are described above in connection with FIG. 2.

The suspension device listens or a telemetry signal from the implanted device (126). When no such signal is received, the suspension device continues to indicate that there has been no confirmation that the function of the implanted device has been suspended (134). When a telemetry signal is received, the suspension device validates the signal (128).

When the telemetry signal does not include confirmation that the function of the implanted device has been suspended, the suspension device continues to indicate that there has been no such confirmation (134). When the telemetry signal affirmatively confirms that the function of the implanted device has been suspended, however, the suspension device outputs an indication that the function has been suspended (132). In the embodiment shown in FIG. 4, suspension device 70 would provide confirmation of suspension by illuminating green LED 94.

After outputting an indication that the function has been suspended (132), suspension device continues to listen for the telemetry signal (126). When the telemetry signal is no longer received, the suspension device indicates that there is no confirmation that the function is suspended (134). Alternatively, when the implanted device's telemetry signal, following validation (128), does not acknowledge suspension of the function, the suspension device likewise indicates that there is no confirmation that the function is suspended (134).

In this way, the suspension device advantageously implements a “fail safe” technique. An indication that the function is suspended is output when there is an affirmative acknowledgment in the telemetry signal of the implanted device that the function is indeed suspended (132). Otherwise, the suspension device outputs an indication that the function is not suspended (124, 134).

When it is no longer desired for the function of the implanted device to be suspended, the suspension device may be moved away from the patient. This may cause the suspension device to receive a telemetry signal that the function has been automatically re-enabled, or it may cause the suspension device to lose the telemetry signal. In either case, the suspension device outputs an indication that the function is not suspended (134).

The suspension device may continue to listen for the telemetry signal even after being removed from the patient (126). In a typical application, the suspension device will continue to listen for a period of time (136). When there is no telemetry signal for that period of time, the suspension device may time out and shut down (138).

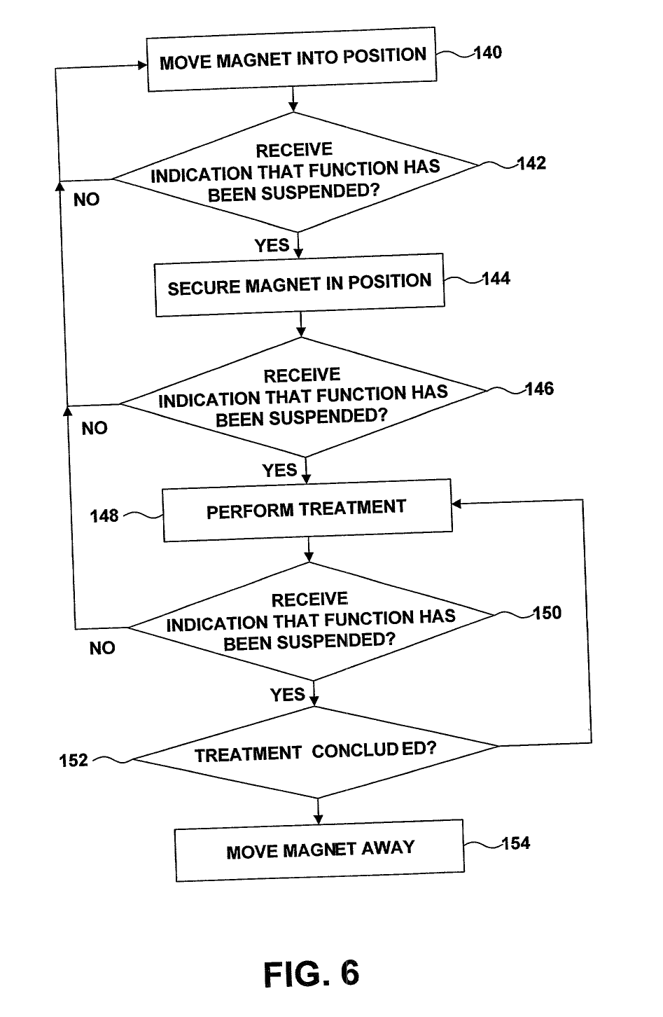

FIG. 6 is a flow diagram illustrating another embodiment of the invention. An operator places the suspension device on the patient, and moves the magnet into position relative to the patient's implanted device (140). The operator checks to see whether there is an indication that the function of the implanted device has been suspended (142). If there is no such indication, the operator may move the magnet (140) until there is such indication (142).

Once there is an indication that the function of the implanted device has been suspended, the operator knows that function is suspended. If the function is responsive to the state of a magnetically operated switch, the operator knows that the magnetically operated switch is in a state that suspends the function. The operator may secure the position (144) of the suspension device and/or the magnet so that they do not move relative to the patient. The operator may, for example secure the device and/or magnet in place with adhesive tape.

While there is an indication that the function of the implanted device has been suspended (146), treatment (148), such as an electrocautery procedure, may be performed. The operator of the suspension device may or may not participate in this treatment. If, in the course of the treatment, the suspension device indicates that the function is not suspended (150), the operator may reposition the magnet (140) until there is such indication (142). When the procedure is completed (152), the operator simply moves the magnet away from the patient (154). The suspended function of the implanted device is automatically re-enabled.

As shown in FIG. 6, the suspension device has the advantage of being easy to operate. In the case of suspension device 70 shown in FIG. 4, the operator merely needs to move the suspension device and magnet relative to the patient until green LED 94 is illuminated. When suspension is no longer required, the suspension device and magnet are removed. The operator does not need to be a specialist to operate suspension device 70.

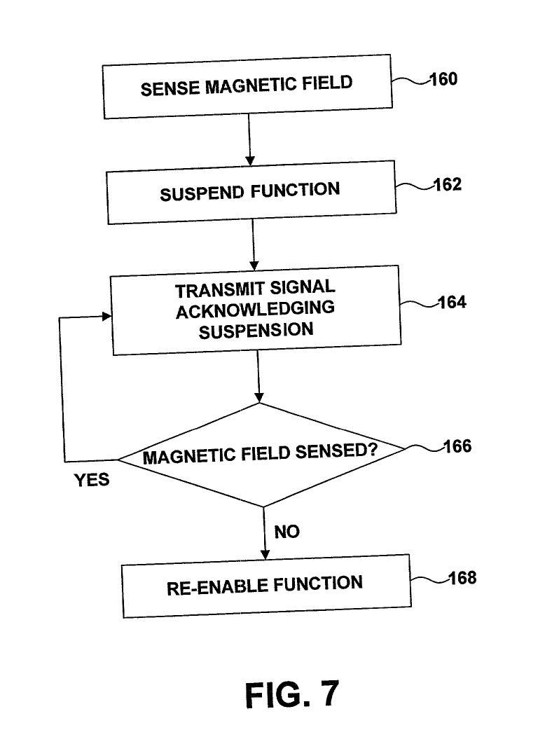

FIG. 7 is a flow diagram example mode of operation of an implanted device according to the principles of the invention. The device senses a magnetic field (160), such as by using a magnetically operable switch. In response to the magnetic field, the device suspends a function (162). The device further responds to the magnetic field by transmitting a signal that the function has been suspended (164).

The device continues to monitor for the presence of the magnetic field (166). As long as the device senses the magnetic field, the device transmits the signal (164). When the magnetic field is not present or no longer strong enough to be sensed, the device re-enables the function (168). Optionally, the device may further transmit a signal that the function has been re-enabled.

Various embodiments of the invention have been described. These embodiments are illustrative of the practice of the invention. Although described in detail in connection with an ICD, the invention may find application with other implantable devices that suspend a function in the presence of a magnetic field and generate a signal when the function is suspended. Moreover, the suspension device is not limited to surgical contexts. For example, a patient receiving inappropriate shocks from an ICD may use a suspension device to suspend the shocks until the ICD can be reprogrammed with a programmer.

Various modifications to the apparatus or methods may be made without departing from the scope of the invention. For example, the suspension device may be equipped with an off switch, such that the suspension device may be affirmatively deactivated rather than waiting to time out. The suspension device may include more or fewer indicators than described herein. The suspension device may be powered by a rechargeable cell rather than by batteries. These and other embodiments are within the scope of the following claims.

1. A method comprising:
emitting a magnetic field to suspend a function of a device implanted in a body;
receiving a signal that the function has been suspended; and
outputting an indicator that the function has been suspended.

2. The method of claim 1, further comprising validating the received signal.

3. The method of claim 1, further comprising outputting a second indicator that the function of the implanted device is not suspended when the signal is no longer received.

4. The method of claim 1, wherein outputting an indicator comprises illuminating a light.

5. The method of claim 1, wherein outputting an indicator comprises sounding an audible tone.

6. The method of claim 1, wherein the function of the implanted device comprises detection of cardiac signals.

7. The method of claim 1, wherein receiving a signal that the function has been disabled comprises receiving a signal from the implanted device.

8. A method comprising:
placing a first device proximal to a second device, the first device including a magnet and the second device implanted in a body of a patient; and
receiving an indication from the first device that a function of the implanted device has been suspended.

9. The method of claim 8, further comprising performing treatment on the patient when the indication is received.

10. The method of claim 8, further comprising:
moving the first device away from the second device; and
receiving an indication from the first device that the function of the implanted device is not suspended.

11. The method of claim 8, further comprising magnetically operating a switch that suspends the function.

12. The method of claim 8, further comprising:
receiving an indication from the first device that the function of the implanted device is not suspended;
moving the first device relative to the second device; and
receiving an indication from the first device that the function has been suspended.

13. A device comprising:
a magnet that produces a magnetic field;
a receiver that receives a signal when an implanted device suspends a function in response to the magnetic field; and
an indicator controlled by the receiver according to the signal.

14. The device of claim 13, wherein the receiver activates the indicator upon receiving the signal.

15. The device of claim 13, wherein the indicator comprises a light emitting diode.

16. The device of claim 13, wherein the indicator comprises a tone generator.

17. The device of claim 13, wherein the magnet comprises a permanent magnet.

18. The device of claim 13, wherein the receiver includes a signal processor comprising:
an amplifier;
a filter to receive an output from the amplifier;
a signal detector to receive an output from the filter; and
a signal validator coupled to the signal detector.

19. The device of claim 13, further comprising a processor coupled to the receiver.

20. The device of claim 13, further comprising an antenna coupled to the receiver.

21. The device of claim 13, further comprising a second indicator, wherein the receiver activates the second indicator when not receiving the signal.

22. A device comprising:
a magnet;
a receiver;
a first indicator that activates when the receiver receives a signal that an implanted device suspends a function;
a second indicator that activates when the receiver does not receive a signal that an implanted device suspends a function; and
a power supply that powers the receiver and the first and second indicators.

23. The device of claim 22, further comprising a third indicator that activates when the power level of the power supply is low.

24. The device of claim 22, further comprising a magnet housing secured inside the device, the magnet disposed in the magnet housing.

25. The device of claim 22, wherein the power supply comprises batteries.

26. A method comprising:
sensing a magnetic field;
suspending a function; and
transmitting a signal that the function has been suspended in response to the magnetic field.

27. The method of claim 26, further comprising:
discontinuing sensing the magnetic field; and
re-enabling the suspended function in response to the discontinuation of sensing of the magnetic field.

28. The method of claim 27, further comprising transmitting a signal that the suspended function has been re-enabled in response to the discontinuation of sensing of the magnetic field.
